(19) United States
(12) Patent Application Publication
(10) Pub. No.: US 2003/0077379 A1
(43) Pub. Date: Apr. 24, 2003

(54) Safe touch window for enclosure

(76) Inventors: Terence P. Roberts, Burnsville, MN (US); Gary Mercier, Tucson, AZ (US)

Correspondence Address: Nelson R. Capes, BRIGGS AND MORGAN, 2400 IDS Center, 80 South Eighth Street, Minneapolis, MN, 55402, US

(21) Appl. No.: 10/090,648

(22) Filed: Mar. 4, 2002

Related U.S. Application Data
(60) Provisional application No. 60/272,936, filed on Mar. 2, 2001.

(51) Int. Cl. A61B 5/103

(52) U.S. Cl. 427/1

(57) ABSTRACT

Apparatus for providing purge gas protection to equipment requiring operator touch access. The apparatus includes:

a) a frame engaging the equipment to be protected;

b) a source of purging gas connected to the frame and an outlet in the frame, the purging gas passing through the outlet to the ambient environment;

c) a flexible window mounted on the frame, the window having sufficient flexibility as to allow an operator to press the window against the protected equipment as to activate the equipment; and

d) a detector signaling loss of purging gas pressure within the frame.

The frame may be electrically conductive so that it acts as a spark arrestor.

### BACKGROUND OF THE INVENTION

This is a continuation of U.S. Provisional Patent Application Serial No. 60/272,936, filed Mar. 2, 2002.

The present invention is directed to an apparatus for providing purge gas protection to equipment that requires operator touch access.

This product is intended as an enhancement for electrical devices protected by a manner of purging and pressurization as defined in NFPA 496 and similar related standards. Purge and Pressurization with air or inert gas is an internationally accepted approach for protecting equipment in a hazardous area. The other two standard approaches are explosion proof enclosures for the protected equipment and intrinsic safe interface for the said equipment. This design is limited to purge protection primarily by providing a flexible secondary cavity in front of certain devices that need touch access for their operation. As an enhancement, we add the possibility of intrinsic safe touch screen feature added to this design. This addition would be used in such a manner that a rupture in the touch screen would be detected by action of the signal from the touch screen surface. However, the primary design feature is detection of loss of pressure if the touch window is ruptured without adding to the reduction of safety in the main enclosure by a serious pressure loss therein and without adding to the possible means of sparks exiting by a pathway from inside the protected enclosure through the touch window cavity.

This is accomplished by use of a spark arresting framework that also acts as an acceptable purge gas path without allowing for so large a flow as to allow significant pressure loss in the main enclosure.

### SUMMARY OF THE INVENTION

Apparatus for providing purge gas protection to equipment requiring operator touch access, the apparatus comprising:

a) a frame engaging the equipment to be protected;

b) a source of purging gas connected to the frame and an outlet in the frame, the purging gas passing through the outlet to the ambient environment;

c) a flexible window mounted on the frame, the window having sufficient flexibility as to allow an operator to press the window against the protected equipment as to activate the equipment;

d) a detector signaling loss of purging gas pressure within the frame; and

e) wherein the frame is electrically conductive whereby the frame acts as a spark arrestor.

A principal object and advantage of the present invention is that it provides purge gas protection to equipment that needs touch access from the operator.

Another principal object and advantage of the present invention is that it may include a detector that signals the loss of purging gas pressure within it, thereby allowing safe shut-down of the equipment.

Another principal object and advantage of the present invention is that it may provide a frame that is electrically conductive and when grounded provides spark arrest protection to the equipment.

### BRIEF DESCRIPTION OF THE DRAWINGS

FIG. 1 is a perspective view of the present invention.

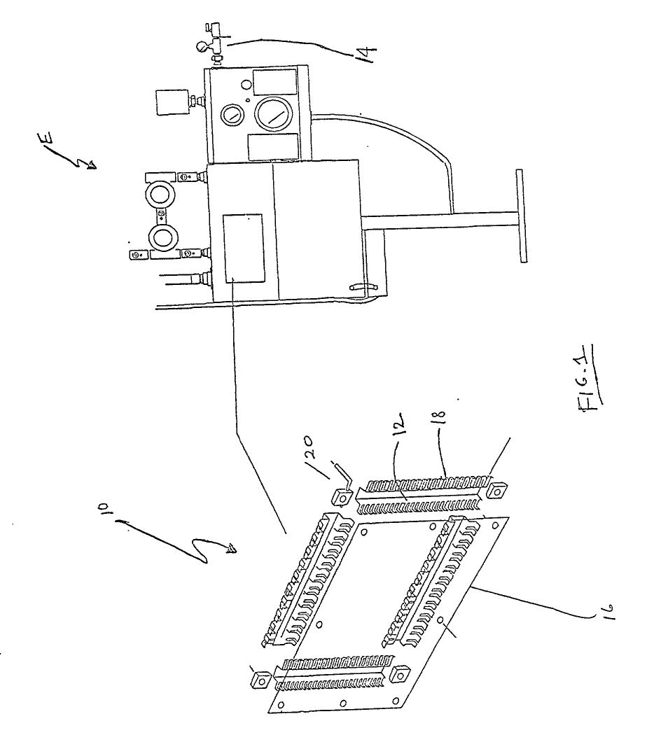

### DETAILED DESCRIPTION OF THE PREFERRED EMBODIMENT

The apparatus of the present invention is generally shown in the Figures. as reference numeral 10.

The apparatus 10 is intended to mount to or otherwise engage equipment E that requires purge gas protection.

The apparatus 10 comprises a frame 12 engaging the equipment E; a source 14 of purging gas connected to the frame 12; and a flexible window 16 mounted on the frame 12, the window having sufficient flexibility as to allow an operator to press the window 16 against the protected equipment E as to activate the equipment.

The apparatus 10 may further comprise an outlet 18 in the frame 12, the purging gas passing through the outlet to the ambient environment.

The apparatus 10 may further comprise a detector 20 signaling loss of purging gas pressure within the frame 12. The detector 20 may either sense a loss of purging gas pressure within the frame 12 or a loss of integrity in the window 16.

The frame 12 may be electrically conductive and grounded in order to serve as a spark arrestor for the equipment E.

Window material may be any transparent material that is movable and flexible enough to allow the window to be pushed inwards until it touches the operator panel enclosed within. The frame gap is such that the window may be pushed in to complete the operation of the protected touch panel on the protected device. The window is such thickness that only one pushbutton on the device will be activated. If the window is too thick, multiple buttons may be pressed at once on the protected device. The window material may be any polycarbonate or plastic or fiberglass or any flexible transparent material. The mil thickness will vary with the application but is typically three to ten mils with present day materials.

The frame may be aluminum foam or metal fingers or metal bars with small holes or any metal foam or metal wool material encased in any open framework that allows air to penetrate to the metal wool. Any frame having small holes to allow limited air flow and to limit the ability for sparks to be transmitted through the frame may be used. This includes the use of sintered metal products. The frame may exhibit flame arresting capabilities if the opening size is sufficiently small. The frame may involve the use of screen material.

Metal or electrical conductive frames have electrical advantages for grounding but this patent included the use of non-metallic materials like ceramic foam or porous ceramic materials.

Conductive plastics that are also porous may be used.

Actual combined opening of the porous media is such that the total square mm of effective open area is at or above a ratio to the volume enclosed by the protective window. That is for every so many cubic meters there is a minimum square meter total effective open area along the frame circumference to satisfy purging requirements.

Flexible window material is in practice chosen for being resistant to solvents and puncture resistant while being thin enough to be flexible and being flexible enough to avoid multiple push button activation when attempting to push one button.

This can be used for push buttons or touch pads or touch and slide operators or any type operator that by its design is not intrinsically safe and cannot be purged from behind when surface mounted as part of an electrical appliance.

This design pushes the appliance back in the protected region and provides a primary but isolated pressurized and pressure safetied region. The air pressure through the purgable frame forces the flexible window out.

Touching the window forces the window in.

The force required to move the touch window is insignificant in comparison to the force to activate any typical touch pad or touch button device.

This application typically puts less than one inch of water pressure and by code should never exceed ten inches of water pressure. However, the patent does not limit the pressure range of operation.

The Safe Touch is a product to allow membrane devices including but not limited to reorders or operator interface devices or control devices or alarm devices or microprocessor or computer devices having membrane type key pads and or displays including those of polycarbonate or other material composition that by their nature are 1) Not intrinsically safe as defined in the electric code or by electrical associations regard being placed and operated in a hazardous area as so defined by the same institutions and codes of such institutions; 2) Is not purgable as defined by NFPA 496 and ISA Purge Standards and IEEE or UL or FM or other approval or testing agencies in that the electrical conductors therein are not open to the purge gas present in the enclosure said device is mounted in. Therefore, said devices have exposed surfaces in the hazardous environment that are not intrinsically safe and are not protected by the purging gas provided.

The Safe Touch product allows mounting the product so it sits back in the protected enclosure with a flexible transparent window material provided in the opening in front of the so protected appliance or device such that said device may be operated by means of pushing the flexible window and thereby touching and pushing on the membrane switch operators. This allows protective gas to surround the device instead of having the membrane surface exposed to hazard.

As a further safety enhancement, this design allows purge gas to circulate in the operator area without adding to the possibility of loosing protective gas pressure on the other components mounted in the protected enclosure if the touch window is ruptured. This is accomplished by providing a pressure sensing element in the window area which will detect a drop in operating pressure if sufficient window fault occurs.

Said window frame is flow restrictive. This allows detection of pressure loss in touch window area before the entire enclosure looses pressure.

Said window frame is metallic and is therefore groundable. This enhances the design.

Said window frame may be non-metallic and flow restrictive as an alternative but a less good design.

Said window frame serves as a spark arresting device regards electrical failures that may occur from switching apparatus therein, preventing a clear path of egris for hot embers from failed electrical devices.

This product allows purge gas to flow over a non purgable membrane surface. This product allows the above with the advantage of spark arresting material to provide a path for protective purge gas from the main enclosure. This spark arresting frame prevents loss of protective gas pressure in the main enclosure if the movable transparent window is ruptured. The rupture of the movable transparent window is detected by a pressure switch or pressure transducer or similar device monitoring the pressure in the cavity between the framing and the flexible touch window. If this pressure is lost by means of failure of the flexible window, then the pressure switch or transducer causes the equipment to safely be de-energized.

An additional means of safety can be the addition of a thing conductive serpentine conductor place over the entire active portion of the flexible touch window such that any hole larger in diameter than the cross-section of the conductor will cause the conductor path to open and act as a safety switch. An additional means could be any of the touch screen analog or capacitive designs that could be used to detect screen touch. However, such design is always used in conjunction with a rated intrinsic safe interface device to allow the conductor paths on the touch screen to be active in the hazardous area.

The present invention may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the invention.

What is claimed:

1. Apparatus for providing purge gas protection to equipment requiring operator touch access, the apparatus comprising:
a) a frame engaging the equipment to be protected;
b) a source of purging gas connected to the frame; and
c) a flexible window mounted on the frame, the window having sufficient flexibility as to allow an operator to press the window against the protected equipment as to activate the equipment.

2. The apparatus of claim 1, further comprising an outlet in the frame, the purging gas passing through the outlet to the ambient environment.

3. The apparatus of claim 1, further comprising a detector signaling loss of purging gas pressure within the frame.

4. The apparatus of claim 3, wherein the detector measures loss of gas pressure within the frame.

5. The apparatus of claim 3, wherein the detector senses an interruption in the surface of the window.

6. The apparatus of claim 1, wherein the frame is electrically conductive whereby the frame acts as a spark arrestor.

7. Apparatus for providing purge gas protection to equipment requiring operator touch access, the apparatus comprising:
a) a frame engaging the equipment to be protected;
b) a source of purging gas connected to the frame and an outlet in the frame, the purging gas passing through the outlet to the ambient environment;
c) a flexible window mounted on the frame, the window having sufficient flexibility as to allow an operator to press the window against the protected equipment as to activate the equipment; and
d) a detector signaling loss of purging gas pressure within the frame.

8. The apparatus of claim 5, wherein the detector measures loss of gas pressure within the frame.

9. The apparatus of claim 5, wherein the detector senses an interruption in the surface of the window.

10. The apparatus of claim 1, wherein the frame is electrically conductive whereby the frame acts as a spark arrestor.

11. Apparatus for providing purge gas protection to equipment requiring operator touch access, the apparatus comprising:
a) a frame engaging the equipment to be protected;
b) a source of purging gas connected to the frame and an outlet in the frame, the purging gas passing through the outlet to the ambient environment;
c) a flexible window mounted on the frame, the window having sufficient flexibility as to allow an operator to press the window against the protected equipment as to activate the equipment;
d) a detector signaling loss of purging gas pressure within the frame; and
e) wherein the frame is electrically conductive whereby the frame acts as a spark arrestor.

12. The apparatus of claim 9, wherein the detector measures loss of gas pressure within the frame.

13. The apparatus of claim 9, wherein the detector senses an interruption in the surface of the window.
